(19) United States
(12) Patent Application Publication
(10) Pub. No.: US 2019/0091251 A1
(43) Pub. Date: Mar. 28, 2019

(54) METHOD FOR INCREASING ABUNDANCE OF LACHNOSPIRA

(71) Applicant: TATA CHEMICALS LIMITED, Mumbai (IN)

(72) Inventors: Anirban Bhaduri, Pune (IN); Ashok Kumar Dubey, Pune (IN); Arup Basu, Pune (IN)

(21) Appl. No.: 16/140,598

(22) Filed: Sep. 25, 2018

(30) Foreign Application Priority Data
Sep. 25, 2017 (IN) 201721033942

(51) Int. Cl. A61K 31/702; A61K 9/00

(52) U.S. Cl. CPC A61K 31/702; A61P 1/00; A61K 9/0053

(57) ABSTRACT

The present application provides a method of increasing abundance of Lachnospira in gastro intestinal tract of a subject. The method comprising administering to said subject at least 5 grams per day of fructo-oligosaccharide, such that the abundance of Lachnospira is increased to a statistically significant level when measured by F-test. A composition for increasing abundance of Lachnospira in gastro intestinal tract of a subject is also provided.

### FIELD OF THE INVENTION

The present disclosure relates to a method and composition for promoting growth of a bacteria in the intestinal tract of a subject. Specifically, the disclosure relates to a method and composition for increasing the abundance of the bacteria Lachnospira in the gastro intestinal tract of a mammal.

### BACKGROUND

The gastro intestinal tract or gut of a mammal harbors within itself a complex ecosystem that includes several bacterial species. For example, there are approximately 300 to 500 bacterial species in the intestinal tract of a human being (Quigley E M M. Gut Bacteria in Health and Disease. Gastroenterology & Hepatology. 2013; 9(9):560-569). These bacteria are extremely beneficial to the host. For example, they help in maintaining a healthy gut and increase resistance against pathogenic bacteria.

Lachnospira a genus belonging to the Lachnospiraceae family is are anaerobic bacteria classified under the taxonomic order Clostridiales. These bacteria are commonly reported in mammal gut and help in maintaining gut homeostasis and epithelial integrity due to their ability to biochemically synthesize butyrate (Vital et al., 2014). Lachnospira are also reported to suppress opportunistic pathogen, Clostridium difficile (Reeves et al., 2012).

Reduced levels of Lachnospira bacteria are observed in patients that have Parkinson's disease (Hill-Burns et al., 2017). Reduced level of these bacteria is also observed in patients that have colorectal cancer. It is reported that a 1.2 fold reduction is observed in patients with convention adenoma (colorectal cancer) and 1.52 fold reduction is observed in patients with hyperplastic polyp (colorectal cancer) (Peters et al., 2016). Thus, Lachnospiraceae has a protective effect against colorectal cancer (Peters et al 2016). Further, these bacteria are reported to negatively correlate with visceral fat mass (VFM) and android/gynoid ratio (measures of body fat distribution), suggesting a potential protective role for these bacteria in cardiovascular risk (Beaumont et al., 2016)

Thus, for certain conditions there is a need for enhancing the population of Lachnospira in the gastro intestinal tract of a mammal especially a human being.

### SUMMARY

A method of increasing abundance of Lachnospira in gastro intestinal tract of a subject is provided. The method includes administering to said subject at least 5 grams per day of fructo-oligosaccharide, such that the abundance of Lachnospira is increased to a statistically significant level when measured by F-test.

A composition for increasing abundance of Lachnospira in gastro intestinal tract of a subject is also provided. The composition comprises at least 5 grams of fructo-oligosaccharide, wherein said composition promotes an increase in abundance of Lachnospira in the gastro intestinal tract of the subject to a statistically significant level when measured by F-test.

### BRIEF DESCRIPTION OF DRAWINGS

FIG. 1 depicts a sample processing pipeline for microbiome annotation in accordance with an embodiment.

FIG. 2 depicts the distribution of Lachnospira 97 otu55 in fecal sample before and during fructo-oligosaccharide intervention at a dose of 5 grams of fructo-oligosaccharide per day in accordance with an embodiment.

FIG. 3 depicts the distribution of Lachnospira unclassified in fecal sample before and during fructo-oligosaccharide intervention at a dose of 5 grams of fructo-oligosaccharide per day in accordance with an embodiment.

FIG. 4 depicts the distribution of Lachnospira 97otu7804 in fecal sample before and during fructo-oligosaccharide intervention at a dose of 10 grams of fructo-oligosaccharide per day in accordance with an embodiment.

FIG. 5 depicts the relative abundance of Roseburia hominis in fecal samples before and during fructo-oligosaccharide intervention at a dose of 10 grams per day.

FIG. 6 depicts the relative abundance of Roseburia hominis in fecal samples before and during fructo-oligosaccharide intervention at a dose of 5 grams per day.

FIG. 7 depicts relative adundance of Roseburia hominis in fecal samples before and during fructo-oligosaccharide intervention at a dose of 2.5 grams per day.

FIG. 8 depicts relative abundance of Bacteroides plebeius in fecal samples before and during fructo-oligosaccharide intervention at a dose of 10 grams per day.

FIG. 9 depicts relative abundance of Bacteroides plebeius in fecal samples before and during fructo-oligosaccharide intervention at a dose of 5 grams per day.

FIG. 10 depicts the relative abundance of Bacteroides plebeius in fecal samples before and during fructo-oligosaccharide intervention at a dose of 2.5 grams per day.

FIG. 11 depicts the relative abundance of Prevotella copri in fecal samples before and during fructo-oligosaccharide intervention at a dose of 10 grams per day.

FIG. 12 depicts the relative abundance of Prevotella copri in fecal samples before and during fructo-oligosaccharide intervention at a dose of 5 grams per day.

FIG. 13 depicts the relative abundance of Prevotella copri in fecal samples before and during fructo-oligosaccharide intervention at a dose of 2.5 grams per day.

### DETAILED DESCRIPTION

For the purpose of promoting an understanding of the principles of the invention, reference will now be made to embodiments and specific language will be used to describe the same. It will nevertheless be understood that no limitation of the scope of the invention is thereby intended, such alterations and further modifications in the disclosed product and process, and such further applications of the principles of the invention therein being contemplated as would normally occur to one skilled in the art to which the invention relates.

It will be understood by those skilled in the art that the foregoing general description and the following detailed description are exemplary and explanatory and are not intended to be restrictive.

Reference throughout this specification to “one embodiment” “an embodiment” or similar language means that a particular feature, or characteristic described in connection with the embodiment is included in at least one embodiment of the present invention. Thus, appearances of the phrase “in one embodiment”, “in an embodiment” and similar language throughout this specification may, but do not necessarily, all refer to the same embodiment.

The present disclosure provides a method of increasing the abundance of bacteria of the genus Lachnospira (hereinafter referred to as Lachnospira) in gastro intestinal tract of a subject. The disclosure also provides a composition for increasing the abundance of Lachnospira in the gastro intestinal tract of a subject.

Increase in abundance of Lachnospira according to the present disclosure means that at least one species of bacteria belonging to the genus Lachnospira, is increased in the gastro intestinal tract of the subject.

The method of increasing the abundance of Lachnospira comprises administering to the subject a composition comprising fructo-oligosaccharide in an amount sufficient to increase the abundance of Lachnospira in the gastro intestinal tract of the subject to a statistically significant level when measured by F-Test.

In accordance with an embodiment, the method results in an increase in absolute abundance of Lachnospira in the gastro intestinal tract of the subject to a statistically significant level when measured by F-Test.

As used herein ‘Absolute Abundance’ refers to the total count of the bacterium of interest. The total count or abundance of a bacterium may be determined most easily by quantification of the bacterium from a sample. Methods for quantification of bacteria include but are not limited to genomic sequencing, metagenomic sequencing or standard cell culturing methods based on optics.

In accordance with an alternative embodiment, the method results in an increase in relative abundance of Lachnospira in the gastro intestinal tract of the subject to a statistically significant level when measured by F-Test.

As used herein ‘Relative Abundance’ refers to the count a bacterium of interest divided by the total count of all the measured bacteria, unless otherwise indicated. The relative abundance of a bacterium may be determined most easily by quantification of all bacteria from a sample. Methods for quantification of bacteria include but are not limited to genomic sequencing, metagenomic sequencing or standard cell culturing methods based on optics.

In accordance with an embodiment, statistically significant level refers to a F-score reflective of a P-value less than or equal to 0.05.

In accordance with an aspect, the subject is a mammal. In accordance with an embodiment the mammal is a human being.

The amount of composition administered to the subject, is such that a dose of at least 5 grams per day of fructo-oligosaccharide is administered to the subject. In accordance with an embodiment, the amount of composition administered is such that the dose of between about 5 grams per day to about 10 grams per day of fructo-oligosaccharide is administered to the subject. The daily amount of fructo-oligosaccharide may be administered for at least about 1 day to about 3 months (i.e., about 90 days). However, the daily amount of fructo-oligosaccharide may be administered even for a longer period. The daily amount may be administered once per day or maybe divided into two or three portions that may or may not be equal.

In accordance with an embodiment, the dose of fructo-oligosaccharide required to increase the absolute abundance or relative abundance of Lachnospira may be determined based on the initial absolute abundance or relative abundance of Lachnospira in the intestine of the subject. For example, on intervention of fructo-oligosaccharide with dosage of 10 grams per day average fold increase across the Lachnospira genus reported was 1.38.

A composition for increasing the growth of Lachnospira in the gastro intestinal tract of the subject is disclosed. The composition comprises at least 5 grams of fructo-oligosaccharide. The composition comprises between 5 grams to 10 grams of fructo-oligosaccharide.

In accordance with an aspect, the composition is a mixture of oligosaccharides consisting of a sucrose molecule (glucose -fructose disaccharide, GF1) linked to one (GF2), or two (GF3) or three (GF4) additional fructose units added by (β-2-1 glycosidic linkages to the fructose unit of the sucrose. The GF2, GF3, and GF4 oligosaccharides, are also called 1-kestose, nystose and fructofuranosyl nystose, respectively.

In accordance with an embodiment, the composition comprises about 40-50% 1-kestose (GF2), 40-50% nystose (GF3) and 5-10% fructofuranosyl nystose (GF4).

The composition may contain other components including but not limited to galacto-oligosaccharides, vitamins, minerals, plant extracts or mixture thereof. The composition may also contain pharmaceutically acceptable excipients.

The composition may be in any desirable formulation suitable for oral intake. In accordance with an embodiment, the composition is in the form of a solid dosage form or a liquid dosage form. In an example, the composition is in the form of an oral syrup.

### EXAMPLES

### Experimental Design and Sample Collection

The study was conducted as per the pertinent requirements of the ICMR guidelines for Biomedical Research on Human Subjects, Good Clinical Practices for Clinical Research in India. The protocol was carried out in accordance with the approved guidelines, and was in agreement with Declaration of Helsinki principles.

The study was a random, double blinded design and was performed on a cohort of sixty (60) subjects. The group of sixty healthy adults were randomly divided into 3 groups: T1 group, T2 group, and T3 group depending upon the amount of fructo-oligosaccharide received/consumed. Table 1, provides the amount of fructo-oligosaccharide that is received/consumed by each of the groups.

| Group | Amount of fructo oligosaccharide consumed per day | |
|---|---|---|
| T1 | 2.5 | grams |
| T2 | 5 | grams |
| T3 | 10 | grams |

All participants consumed the assigned dosage of fructo-oligosaccharide on daily basis for three (3) months. DNA from the gut bacteria present in the fecal samples of the sixty subjects defining the cohort was extracted. For each of the sixty subjects, samples were collected at seven (7) time points—three (3) time points before the subject received fructo-oligosaccharide i.e on the −60^{th}, −30^{th} and −0^{th} day, and 4 times during the three (3) months of intervention of fructo-oligosaccharide i.e., on the 1^{st}, 30^{th}, 60^{th} and 90^{th} day.

### Sample Processing

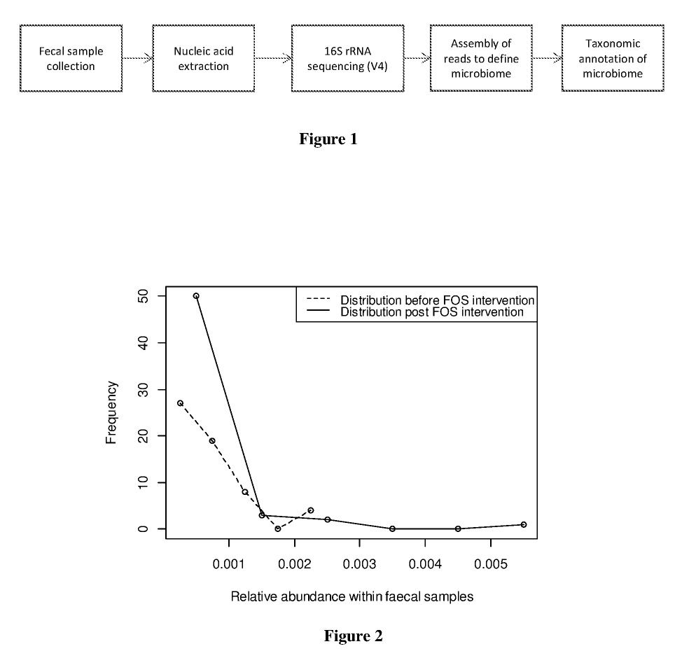

Each fecal sample was processed using standard nucleic acid extraction protocols. After the nucleic acid extraction, 16S rRNA gene based sequencing of the extracted DNA was performed. V4 region of the extracted 16S rRNA gene was amplified and sequenced. Obtained sequenced data was processed using a standard pipeline (using Second Genome pipeline). Post processing through the mentioned pipeline, abundance of Lachnospira was computed and tabulated. An exemplary sample processing pipeline for microbiome annotation is shown in FIG. 1.

### Data Analytics

Statistical assessment of abundance change of microorganism was performed using F-test. The test was performed on the R platform. Change in relative abundance of Lachnospira prior to intervention with fructo-oligosaccharide and during intervention across the study was assessed.

### Results:

It is observed that fructo-oligosaccharide was able to increases counts of Lachnospira. Specifically, the study explored relative abundance change for three (3) Lachnospira species (Lachnospira 97otu7804, Lachnospira unclassified and Lachnospira 97otu55). This change was observed most optimally at a dosage of 5 grams per day. Significant increase in relative abundance was observed across the dosages and is summarized in Table 1.

TABLE 1
F-test statistic (and P-value) to report increase of Lachnospira
species in individuals intervened with fructo-oligosaccharide.

| | Dosage (grams per day) | | |
|---|---|---|---|
| Organisms | 2.5 | 5 | 10 |
| Lachnospira 97otu55 | 1.4(0.2) | 0.5(0.02) | 1.7(0.05) |
| Lachnospira unclassified | 3.7(2.9*e−6) | 0.7(0.1) | 0.3(8.6*e−7) |
| Lachnospira 97otu7804 | 0.66(0.1) | 2.0(0.01) | 0.2(3.5*e−11) |

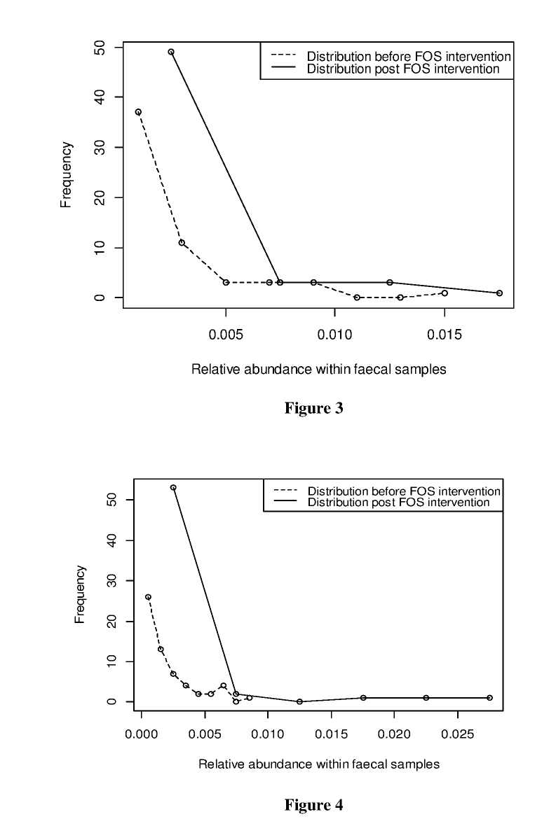

FIG. 2-4 reports increase of different species of Lachnospira on intervention with fructo-oligosaccharide.

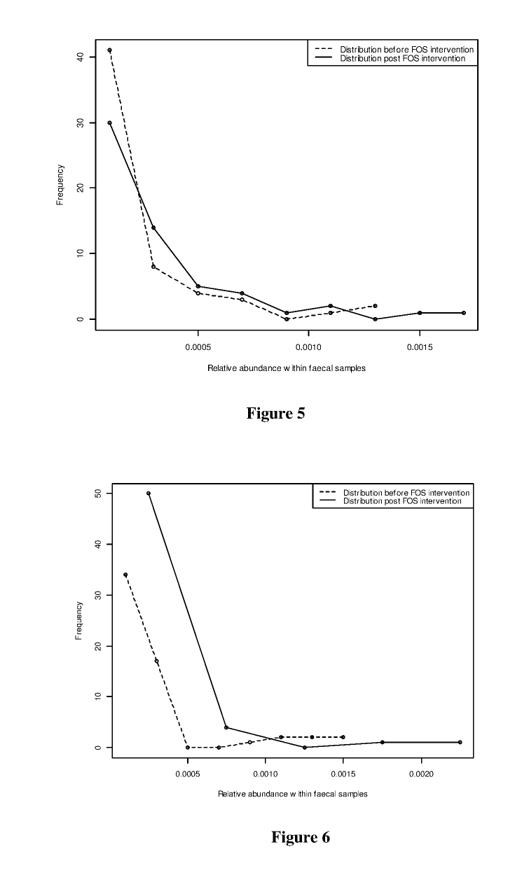

However, no change in relative abundance of other bacterium such as Roseburia hominis, Prevotella copri and Bacteroides plebeius was observed in subjects after intervention of fructo-oligosaccharide. FIGS. 5-13 report the relative abundance of some of these bacteria before and during intervention with fructo-oligosaccharide.

FIG. 5 depicts the relative abundance of Roseburia hominis in fecal samples before and during fructo-oligosaccharide intervention. On intervention with fructo-oligosaccharide at a dose of 10 grams per day, no significant relative abundance change was observed (F-statistics=0.64, P-value=0.09).

FIG. 6 depicts the relative abundance of Roseburia hominis in fecal samples before and during fructo-oligosaccharide intervention. On intervention with fructo-oligosaccharide at a dose of 5 grams per day, no significant relative abundance change was observed (F-statistics=1.04, P-value=0.87).

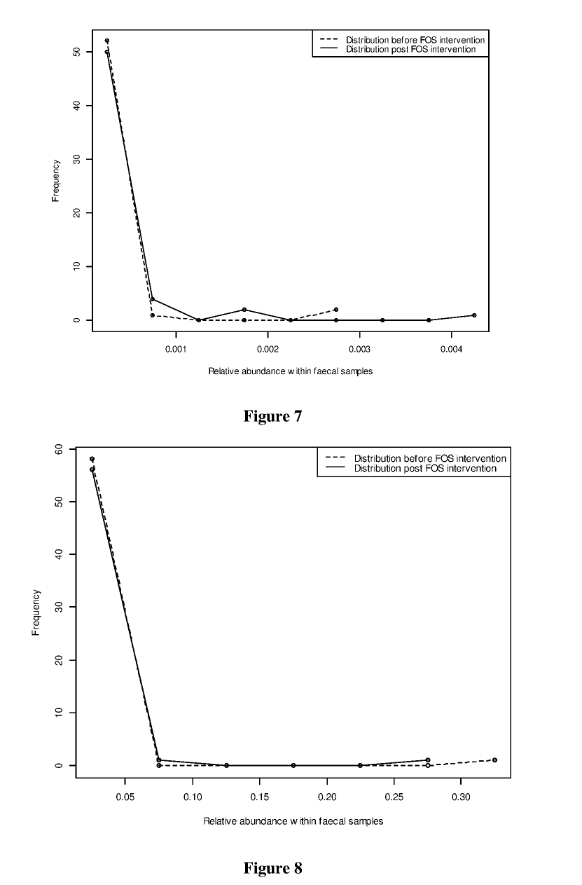

FIG. 7 depicts relative abundance of Roseburia hominis in fecal samples before and during fructo-oligosaccharide intervention. On intervention with fructo-oligosaccharide at a dose of 2.5 grams per day, no significant relative abundance change was observed (F-statistics=0.64, P-value=0.10).

FIG. 8 depicts relative abundance of Bacteroides plebeius in fecal samples before and during fructo-oligosaccharide intervention. On intervention with fructo-oligosaccharide at a dose of 10 grams per day, no significant relative abundance change was observed (F-statistics=0.86, P-value=0.76).

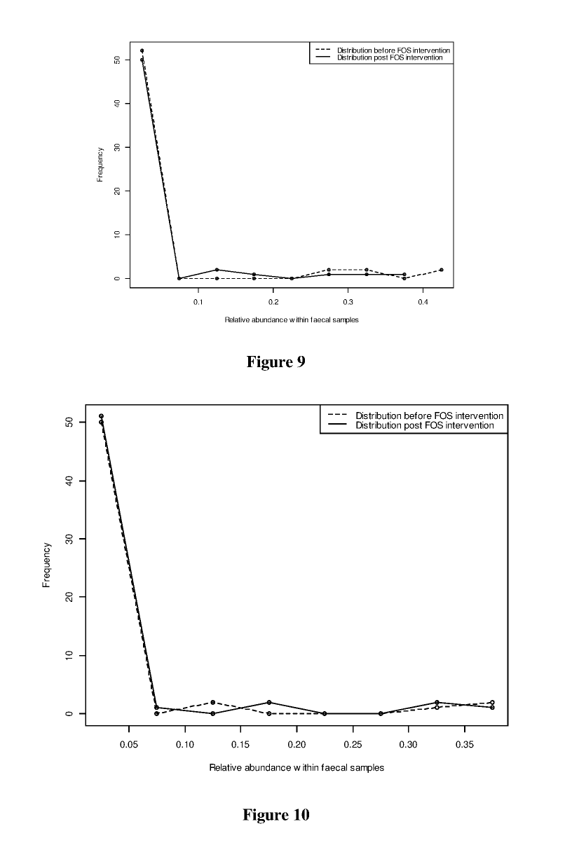

FIG. 9 depicts relative abundance of Bacteroides plebeius in fecal samples before and during fructo-oligosaccharide intervention. On intervention with fructo-oligosaccharide at a dose of 5 grams per day, no significant relative abundance change was observed (F-statistics=1.69, P-value=0.52).

FIG. 10 depicts the relative abundance of Bacteroides plebeius in fecal samples before and during fructo-oligosaccharide intervention. On intervention with fructo-oligosaccharide at a dose of 2.5 grams per day, no significant relative abundance change was observed (F-statistics=1.1, P-value=0.71).

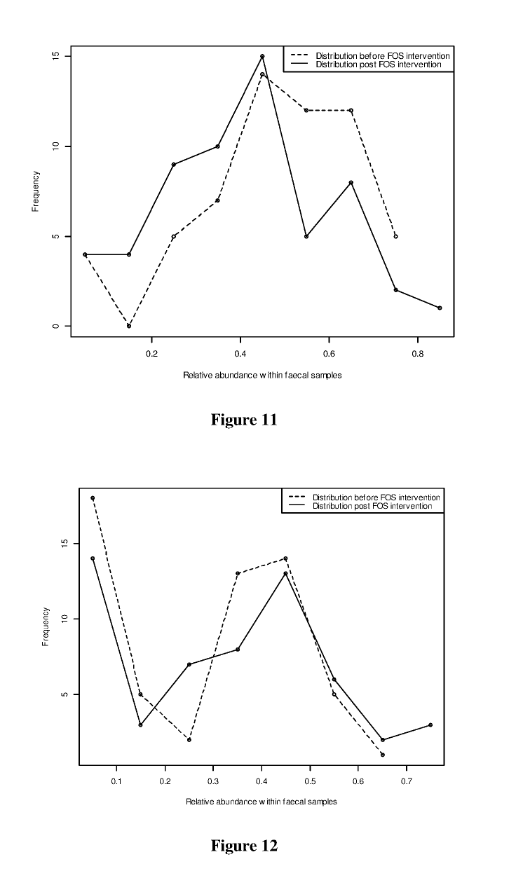

FIG. 11 depicts the relative abundance of Prevotella copri in fecal samples before and during fructo-oligosaccharide intervention. On intervention with fructo-oligosaccharide at a dose of 10 grams per day, no significant relative abundance change was observed (F-statistics=0.95, P-value=0.84).

FIG. 12 depicts the relative abundance of Prevotella copri in fecal samples before and during fructo-oligosaccharide intervention. On intervention with fructo-oligosaccharide at a dose of 5 grams per day, no significant relative abundance change was observed (F-statistics=0.82, P-value=0.44).

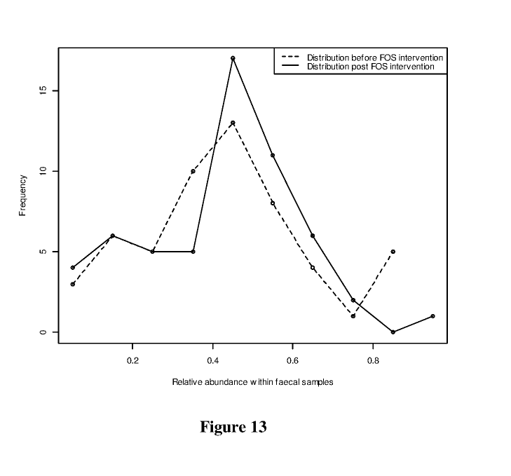

FIG. 13 depicts the relative abundance of Prevotella copri in fecal samples before and during fructo-oligosaccharide intervention. On intervention with fructo-oligosaccharide at a dose of 2.5 grams per day, no significant relative abundance change was observed (F-statistics=1.07 P-value=0.78).

### Specific Embodiments

A method of increasing abundance of Lachnospira in gastro intestinal tract of a subject is disclosed. The method comprising administering to said subject at least 5 grams per day of fructo-oligosaccharide, such that the abundance of Lachnospira is increased to a statistically significant level when measured by F-test.

Such a method, wherein 5 grams per day to 10 grams per day of fructo-oligosaccharide is administered to said subject.

Such a method, wherein the subject is a mammal and preferably the subject is a human being.

Such a method, wherein the fructo-oligosaccharide is administered for 1 to 90 days.

Such a method, wherein the fructo-oligosaccharide administered comprises about 40-50% 1-kestose (GF2), 40-50% nystose (GF3) and 5-10% fructofuranosyl nystose (GF4).

A composition for increasing abundance of Lachnospira in gastro intestinal tract of a subject is disclosed. The composition comprising at least 5 grams of fructo-oligosaccharide, wherein said composition promotes an increase in abundance of Lachnospira in the intestinal tract of the subject to a statistically significant level when measured by F-test.

Such a composition, wherein the composition comprises between 5 grams to 10 grams of fructo-oligosaccharide.

Such a composition, wherein the fructo-oligosaccharide composition comprises about 40-50% 1-kestose (GF2), 40-50% nystose (GF3) and 5-10% fructofuranosylnystose (GF4).

Such a composition, wherein the fructo-oligosaccharide composition is in the form of a liquid or a solid dosage form for oral administration.

Such a composition, wherein the fructo-oligosaccharide composition further comprising other components selected from a group comprising galacto-oligosaccharides, vitamins, minerals, plant extracts, pharmaceutically acceptable excipients or a mixture therefore.

We claim:

1. A method of increasing abundance of Lachnospira in gastro intestinal tract of a subject, the method comprising administering to said subject at least 5 grams per day of fructo-oligosaccharide, such that the abundance of Lachnospira is increased to a statistically significant level when measured by F-test.

2. The method as claimed in claim 1, wherein 5 grams per day to 10 grams per day of fructo-oligosaccharide is administered to said subject.

3. The method as claimed in claim 1, wherein the subject is a mammal and preferably the subject is a human being.

4. The method as claimed in claim 1, wherein the fructo-oligosaccharide is administered for 1 to 90 days.

5. The method as claimed in claim 1, wherein the fructo-oligosaccharide administered comprises about 40-50% 1-kestose (GF2), 40-50% nystose (GF3) and 5-10% fructofuranosylnystose (GF4).

6. A composition for increasing abundance of Lachnospira in gastro intestinal tract of a subject, the composition comprising at least 5 grams of fructo-oligosaccharide, wherein said composition promotes an increase in abundance of Lachnospira in the gastro intestinal tract of the subject to a statistically significant level when measured by F-test.

7. The composition as claimed in claim 6, wherein the composition comprises between 5 grams to 10 grams of fructo-oligosaccharide.

8. The composition as claimed in claim 6, wherein the fructo-oligosaccharide composition comprises about 40-50% 1-kestose (GF2), 40-50% nystose (GF3) and 5-10% fructofuranosyl nystose (GF4).

9. The composition as claimed in claim 6, wherein the fructo-oligosaccharide composition is in the form of a liquid or a solid dosage form for oral administration.

10. The composition as claimed in claim 6, wherein the fructo-oligosaccharide composition further comprising other components selected from a group comprising galacto-oligosaccharides, vitamins, minerals, plant extracts, pharmaceutically acceptable excipients or a mixture therefore.
